(19) United States
(12) Patent Application Publication
(10) Pub. No.: US 2007/0237723 A1
(43) Pub. Date: Oct. 11, 2007

(54) TREATMENT OF COUGH WITH INTERFERON

(75) Inventors: Joseph M. Cummins, Amarillo, TX (US); Martin J. Cummins, Amarillo, TX (US)

Correspondence Address: BARNES & THORNBURG LLP, 11 SOUTH MERIDIAN, INDIANAPOLIS, IN, 46204, US

(73) Assignee: AMARILLO BIOSCIENCES, INC., Amarillo, TX (US)

(21) Appl. No.: 11/693,450

(22) Filed: Mar. 29, 2007

Related U.S. Application Data
(60) Provisional application No. 60/791,060, filed on Apr. 11, 2006.

(51) Int. Cl. A61K 38/21; A61K 9/12

(52) U.S. Cl. 424/45; 424/85.7

(57) ABSTRACT

A method of treating chronic coughing in a patient comprising administering to the patient an effective amount of interferon; a method of treating idiopathic pulmonary fibrosis in a patient comprising orally administering to the patient a composition comprising interferon.

### CROSS-REFERENCE TO RELATED APPLICATIONS

This patent application claims the benefit of U.S. Provisional Patent Application No. 60/791,060, filed Apr. 11, 2006, the content of which is incorporated herein by reference in its entirety.

### BACKGROUND

Coughing is an action that removes irritating substances from the respiratory tract of an individual. Coughs are generally useful and preferably not eliminated. However, in many instances, coughs can be severe enough to impair breathing or prevent rest. This is particularly true for involuntary, frequent or chronic coughing that accompanies an infection or indicates the presence of a disease. In severe cases, prolonged coughing can cause fatigue fractures of lower ribs and costochondritis, an inflammation of the connective tissue between the breastbone and the ribs. Prolonged and forceful coughing can also lead to fainting spells.

Recent reports by the American College of Chest Physicians have confirmed that currently available non-narcotic, over-the-counter cough remedies simply do not work. Accordingly, there is a need for a safe, non-addictive medication that can provide relief to an individual suffering from chronic coughing.

“Interferon” is a term generically describing a distinct group of cytokines exhibiting pleiotropic activity generally categorized as antiviral, antiproliferative and immunomodulatory. In the early years of interferon research, an international committee was assembled to devise a system for orderly nomenclature of interferons and defined “interferon” as follows: “To qualify as an interferon a factor must be a protein which exerts virus non-specific, antiviral activity at least in homologous cells through cellular metabolic process involving synthesis of both RNA and protein.” Journal of Interferon Research, 1, pp. vi (1980). “Interferon” as used herein in describing the present invention shall be deemed to have that definition and shall contemplate such proteins and glycoproteins, including for example, the subtypes interferon-alpha, interferon-beta, interferon-delta, interferon-epsilon, interferon-gamma, interferon-kappa, interferon-lambda, interferon-omega and interferon-tau, regardless of their source or method of preparation or isolation.

Originally identified for their ability to induce cellular resistance to viral infection, interferons are currently known to be potent mediators in the host defense mechanism and homeostasis, modulating both the innate and adaptive immune responses. Interferons are small, inducible, 20-25 KD, usually glycosylated proteins that are produced by vertebrate cells in response to various biological stimuli. Mechanistically, interferons mediate their biological activities by binding to receptors present on the surface of target cells. Specific ligand-receptor interactions trigger intracellular signaling cascade downstream, resulting in the synthesis of proteins that mediate mentioned pleiotropic activities.

Interferons are classified into two groups: type I or type II, based on their structure, physicochemical properties and biological activities. Type I and type II interferons exert their biological effects through different cellular receptors. In mammals, eight families of type I interferon have been described. These are: interferon-alpha, interferon-beta, interferon-delta, interferon-epsilon, interferon-kappa, interferon-lambda, interferon-omega and interferon-tau. Among these families, interferon trophoblast, found only in ruminant ungulates, is not inducible by virus and is produced in the embryonic trophoectoderm at a specific time, early during pregnancy. Its major function is to create conditions for the completion of pregnancy. Interferon-delta, a polypeptide of about 149 amino acids, has been described only in pigs. This interferon is physiologically expressed by trophoblast during the period of implantation in uterus. Interferon-gamma is the sole representative of type II interferon in mammals.

As described herein a method for alleviating chronic coughing that is associated with a pulmonary related disease, disorder, condition, or syndrome is provided. The method comprises administering interferon for contact with the mucosal membranes of the digestive and respiratory tract.

### SUMMARY

A method of treating chronic coughing in an individual is provided. More particularly, a method for alleviating the frequency, intensity or duration of chronic coughing in an individual is provided by administering low dose interferon to the individual. In one embodiment the method comprises treating a chronic cough that is associated with a pulmonary disease or condition. In one embodiment the interferon is either interferon-alpha or interferon-beta and the interferon is administered orally, intranasally or by inhalation. The interferon is administered in an amount effective to diminish at least one cough characteristic including cough duration, cough frequency and cough intensity. In one embodiment the interferon is interferon-alpha and the interferon is administered orally or topically by inhalation in an amount from about 0.1 IU/lb to about 100 IU/lb of patient body weight. When the interferon is administered by inhalation, a metered dose inhaler can be used that provides a dose from about 75 IU to about 1,000 IU of interferon.

As disclosed herein a method is provided for treating idiopathic pulmonary fibrosis. The method comprises orally administering interferon-alpha in an amount effective to diminish a characteristic selected from the group consisting of cough duration, cough frequency and cough intensity.

### BRIEF DESCRIPTION OF THE DRAWINGS

FIG. 1 shows the individual plots representing forced vital capacity (FVC; % predicted) data across time for ten subjects completing 12 months of treatment.

FIG. 2 shows the individual plots representing resting O₂ saturation (%) data across time for nine subjects completing 12 months of treatment.

FIG. 3 shows the individual plots representing post-exercise O₂ saturation (%) data across time for nine subjects completing 12 months of treatment.

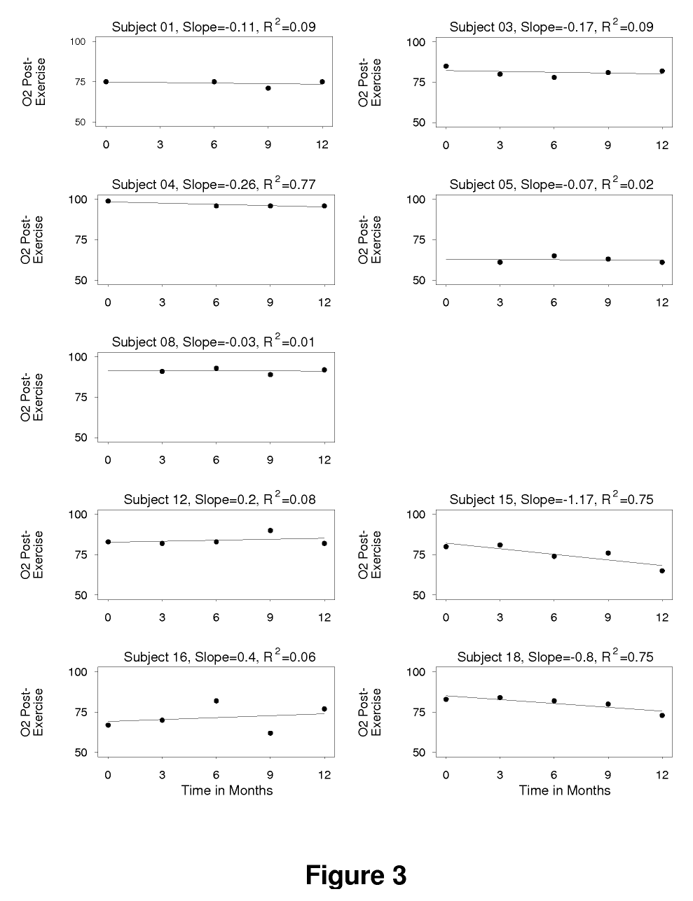

FIG. 4 shows the individual plots representing FVC (% predicted), resting O₂ saturation (%) and post-exercise O₂ saturation (%) data across time for the two subjects treated for 48 and 24 months.

### DETAILED DESCRIPTION

### Definitions

In describing and claiming the invention, the following terminology will be used in accordance with the definitions set forth below.

As used herein, the term “treating” includes prophylaxis of the specific disease, condition, disorder or syndrome, or alleviation of the symptoms associated with a specific disease, condition, disorder or syndrome and/or prevention or elimination of said symptoms.

As used herein, the term “pharmaceutically acceptable carrier” encompasses any of the standard pharmaceutical carriers, such as a phosphate-buffered saline solution, water, and emulsions, such as an oil/water or water/oil emulsion, and various types of wetting agents, and includes agents approved by a regulatory agency of the U.S. Federal Government or listed in the U.S. Pharmacopeia for use in animals, including humans. The term “carrier” refers to a diluent, adjuvant, excipient or vehicle with which an active agent is administered.

### EMBODIMENTS

A method is provided herein for treating persistent and chronic coughing that is associated with an infection, or associated with a pulmonary condition or disease state. The method comprises administering a low dose of interferon to a patient in need thereof. The patient can be any warm-blooded vertebrate, including, but not limited to, a human, a horse, and a dog. In one embodiment the interferon is interferon-alpha or interferon-beta, and more particularly, in one embodiment the administered biologically active interferon is human interferon-alpha. In one embodiment the method comprises treating a chronic cough that is associated with a pulmonary disease or condition/syndrome. In one embodiment the pulmonary disease or condition causing the chronic coughing is Idiopathic Pulmonary Fibrosis (IPF) or Sjogren's syndrome.

The interferon can be administered to the patient through a number of routes, such as orally, bucally, intranasally, intramuscularly, or intravenously. In accordance with one embodiment the interferon is administered orally, intranasally or by inhalation. The interferon-containing composition can be administered in a single dose, or in several doses per day. In accordance with one embodiment the interferon is administered in a form of orally dissolving lozenges.

Interferon of human and murine origin is quantified in the art in terms of International Units (IU). Interferons of other than human or murine origin can be used in accordance with this invention. In that presently accepted practices may not extend the use of “International Units” to quantify non-human and non-murine interferons, it shall be understood that administration of amounts of non-human/non-murine interferons having the same efficacy as the quantities (IU's) of human interferon specified in this description are within the scope of the present invention.

As disclosed herein, a method for treating a patient suffering from chronic coughing is provided wherein a composition comprising interferon is administered to the patient. More particularly, the compositions disclosed herein are used to treat a chronic cough that results from a respiratory infection or is associated with a pulmonary-related disease or condition. In one embodiment the interferon is administered, as needed, to diminish at least one cough characteristic selected from the group consisting of cough duration, cough frequency and cough intensity. In one embodiment the interferon is a type I interferon that is administered orally, in a low dosage form and in one embodiment the interferon is interferon-alpha. In one embodiment, wherein the interferon-containing composition is administered orally, such as bucally, the composition is administered in a form or manner that optimizes contact of the composition with the oral and oral pharyngeal mucosa of the patient. In one embodiment the interferon-containing composition is prepared as a lozenge, powder, liquid or chewable composition.

For the purpose of the present invention, low-dosage, interferon-alpha treatment dosages range from about 0.1 IU/lb to about 100 IU/lb of patient body weight, more typically about 0.5 IU/lb to about 10 IU/lb of patient body weight. Thus, unit dosage forms for human use typically comprise about 5 IU to about 2,500 IU of interferon-alpha, more typically about 10 IU to about 300 IU of interferon-alpha, in combination with a pharmaceutically acceptable carrier therefor. Dosage forms for treatment in accordance with this invention can be in solid, liquid, aerosol, ointment or cream formulation and are typically administered from one to four times daily until the condition being treated is alleviated. In one embodiment, human interferon-alpha is orally administered in a sterile aqueous solution. Chronic administration may be required for sustained benefit. Generally speaking, the dosage forms are administered in a disease state-dependent manner, including particularly administration topically, bucally/sublingually, by oral ingestion or by inhalation, such as through the mouth or nose (i.e., intranasally). In this regard, a metered dose inhaler can be used to administer interferon topically to the cells of the lungs. A metered dose can be from about 75 IU to about 1,000 IU of interferon-alpha.

IPF is a rare, rapidly progressing, interstitial lung disease of unknown origin with marked impairment of quality of life and an expected survival after diagnosis of 3.2 to 5 years. It is characterized by multiple fibroblastic foci with minimal inflammation. A new approach to treating this disease is needed since no therapeutic regimen, including the therapeutic use of steroids, has been demonstrated to be effective.

Although the results of a trial using large dose, injected interferon-gamma have been disappointing, there is a basis for hypothesizing that interferons might be efficacious. Lung biopsy samples have shown that interferon levels are low, while levels of transforming growth factor β1 are high. Interferon-alpha can increase production of interferon gamma and aquaporins and inhibit transforming growth factor β1, fibroblast activity, and collagen synthesis.

As described in Example 1, low dose, orally administered interferon-alpha (150 IU three times daily) has been administered to IPF patients and found to have minimal to no side effects. Subjects were evaluated with pulmonary function tests every three months and high resolution computed tomography (HRCT) scans at yearly intervals. Of the 9 subjects who completed at least one year, the forced vital capacity has remained stable in 8 and the oxygen saturation, after a 6-minute walk, has been stable in 7 and improved in 1. One subject showing lack of progression of the disease has been followed for over 4 years and another for 2 years. The 8 subjects whose pulmonary function tests were stable showed no evidence of disease progression on HRCT scans. Most subjects who entered the study with a cough noted marked improvement within the first few weeks of treatment with corresponding increases in quality of life scores. These results strongly suggest that this regimen can prevent progression according to the criteria defined in the International Consensus Statement published by the American Thoracic Society.

In one embodiment a method is provided for treating patients suffering from IPF. The method comprises administering interferon to the patient. In one embodiment the interferon is administered orally and in one embodiment the administered interferon is interferon-alpha, administered in an amount effective to diminish a cough characteristic selected from the group consisting of cough duration, cough frequency and cough intensity. In one embodiment the interferon is administered orally, as a solution, or bucally, using a dissolving lozenge or other slow-release formulation, or by inhalation, using a spray or mist.

### EXAMPLE 1

Treatment of IPF with Orally Administered Interferon-Alpha

IPF is a rare interstitial lung disease of unknown origin for which the average life span has been reported to be between 3.2 and 5 years after diagnosis. Steroids as anti-inflammatory agents are routinely used in treatment, but the International Consensus Statement published by the American Thoracic and British Respiratory Societies in 2000 concluded that there is no objective evidence for any effective treatment, and cited the need for novel approaches including employing antifibrotic agents.

IPF is characterized by fibroblastic foci, often with minimal inflammation, leading to deposition of collagen and rapid destruction of the lung. Transforming growth factor β-1 has been shown to have strong profibrotic activity leading to pulmonary fibrosis, and antagonists of transforming growth factor β1 inhibit myofibroblast differentiation and collagen production. The levels of transcription for the genes for transforming growth factor β1 and connective tissue growth factor are elevated in patients with IPF. Interferons are produced in lung tissue and their levels are low in patients with IPF. Importantly, interferons can inhibit transforming growth factor β1 signaling and the fibrotic response by inhibiting fibroblast proliferation and down-regulating the gene expression for TGF-β1 and procollagen I and III. Interferon-alpha also up-regulates the gene expression of aquaporin-5, which is expressed in the lung and may serve an important function in transpiration.

Interferon-alpha binds to lymphoid and epithelial cells in oral mucosa, is secreted from nasal mucosa during respiratory viral infections and is believed to activate an immunological cascade by interacting with these cells. Given this background, an open-label Phase II trial was conducted administering low-dose (150 IU), orally administered interferon-alpha to patients with advanced IPF.

### Methods

Twenty subjects were enrolled but only eighteen actually received treatment (Table 1). The diagnosis of IPF had been confirmed either by biopsy or HRCT, and all subjects had had significant loss of function as documented by pulmonary function tests (PFTs) by the time they entered this study.

TABLE 1
SUBJECT DEMOGRAPHICS

| | |
|---|---|
| Total starting treatment: | 18 |
| Total treated for 12 months or longer: | 10 |
| Race: | 12 Caucasian; 5 Hispanic; |
| | 1 African-American |
| Ages: | Range = 50–82; Mean = 67 |
| Gender: | 9 females; 9 males |
| Currently receiving treatment: | 7* |
| No longer receiving treatment: | 11 |
| Stopped after 12 months: | 4 |
| Withdrew prior to 12 months: | 4** |
| Died: | 3*** |

*48, 27, 21, 18, 15, 12 & 9 months on treatment by end of March 2006
**One on advice of another physician to begin combined treatment with prednisone and cytoxin, one to enter a transplant program, one to participate in a breast cancer trial and one due to non-compliance in taking the medication.
***Considered not to be adverse reaction to treatment. See details in “Results” section.

A baseline history and physical were performed; blood was drawn for a CBC and Comprehensive Metabolic Panels; and PFTs, chest films and HRCTs were done prior to the start of treatment. Treatment involved the subjects allowing lozenges containing 150 IU of interferon-alpha to dissolve in their mouths three times daily. For the purpose of safety monitoring, subjects were administered the first dose of interferon-alpha under observation in the clinic, a phone call was placed to the subject after 24 hours, and chemistries were taken and the subject seen by a physician in the clinic on days 7, 14, 30 and 60 after the start of treatment. The subjects returned to the clinic every three months for follow-up histories, physicals, chemistries, and PFTs. Chest films and HRCTs were repeated after 12 months. Permission was subsequently obtained to continue subjects on treatment for longer than 12 months if there was evidence of minimal or no progression. However, the first two subjects had stopped medication before permission was received. In response to comments by the subjects, the seven still under treatment were retrospectively given a questionnaire using a 5-point Likert scale to rate various aspects of their IPF-associated cough and its effect on their quality of life prior to treatment and then one month after treatment. The effect of treatment was evaluated on the basis of changes in the ratings.

Assessment of the response to treatment was done using the categories outlined in the International Consensus Statement (2): a) favorable (or improved) status; b) stable (and presumed favorable) response and c) failure to respond on two consecutive visits over a 3- to 6-month period. In this study, the assessment presented involves two of these 6-month periods studied consecutively and uses the limits of the percent change defined under the “stable” category.

### Results

Treatment with interferon-alpha (150 IU tid) was well tolerated with no side effects being reported, consistent with observations in previous trials in other diseases. The death of three subjects was determined to be progression or a complication of severe IPF and not the result of treatment. One patient on interferon-alpha as a “last resort,” entered the hospital after less than 2 months on treatment, withdrew care, and died of respiratory failure; the second, with stable PFTs for 9 months, died after several hospitalizations for pneumonia; and the third with rapidly progressing IPF unresponsive to other therapy died from a pulmonary embolism while hospitalized for unresolving pneumonia.

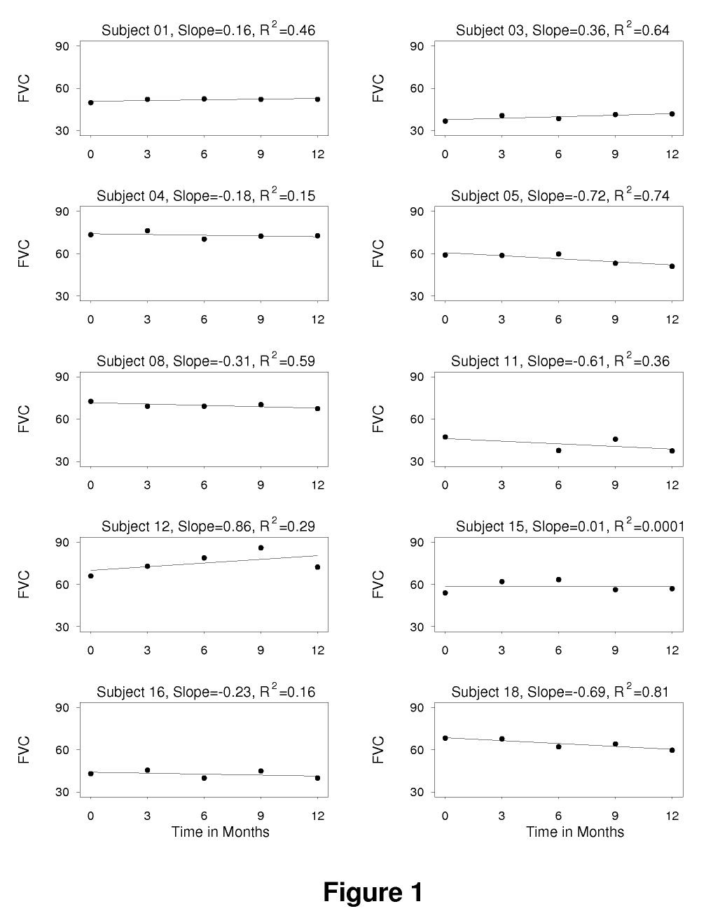

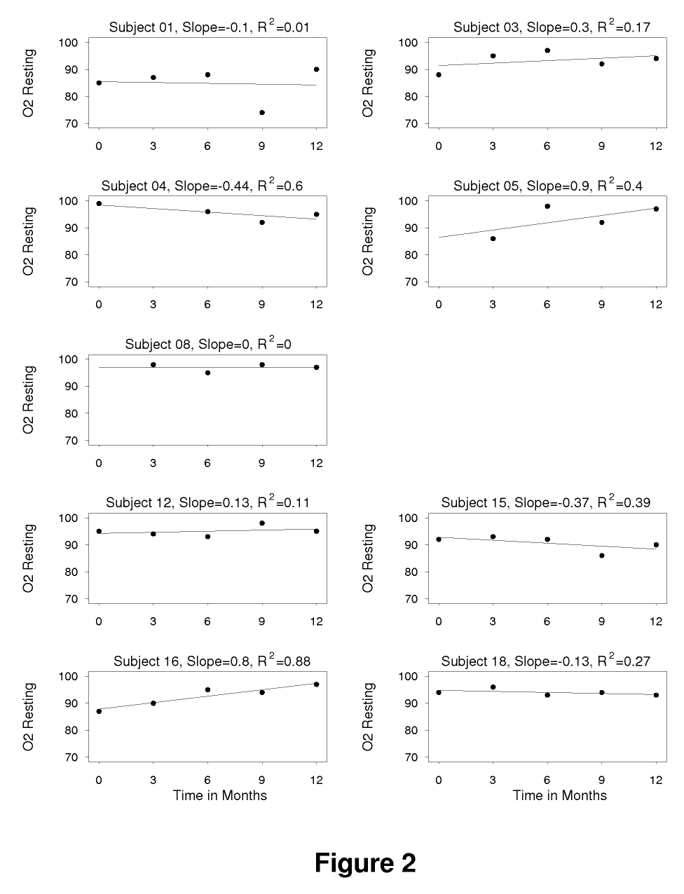

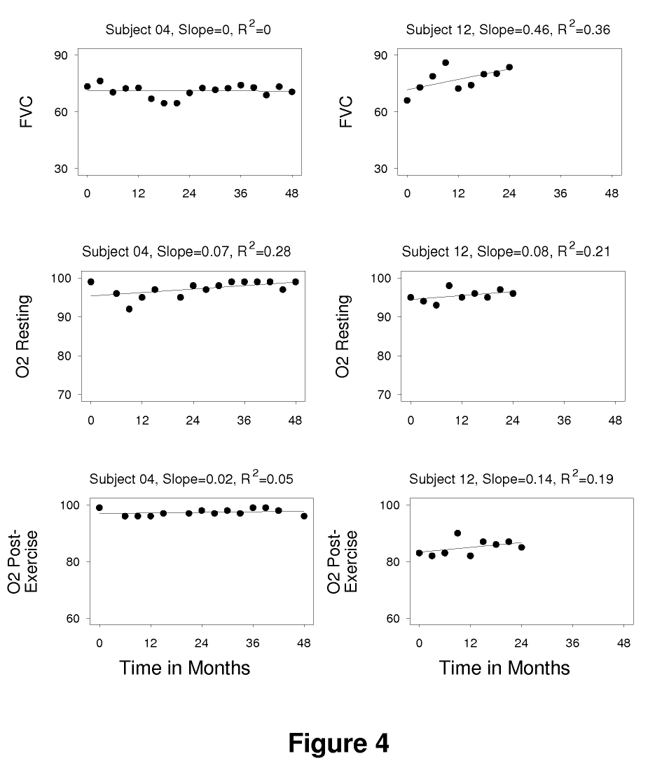

Individual plots across time for FVC, resting oxygen saturation and oxygen saturation following a six-minute walk for each of the ten subjects completing 12 months in the trial are shown in FIGS. 1, 2 & 3, respectively. The slopes for some subjects are positive, and those that are negative are close to zero. The plots for the subjects on the study for two and four years, respectively, show no evidence of progression of the disease (FIG. 4).

The outcomes were assessed by the criteria from the International Consensus Statement across two consecutive 6-month periods. Measurements made at 3-month intervals for FVC (Table 2) were: 2/10 improved; 7/10 subjects stable and 1/10 worse during the first period. Using the 6-month value as a new baseline, 8/10 were stable and 2/10 worse during the second period. For the entire 12-month period, 1/10 was improved, 6/10 were stable and 3/10 were worse.

The outcomes for O₂ saturation post-exercise (Table 3) for the first period were: 1/9 improved; 6/9 stable and 2/9 worse. During the second period, the outcomes were 1/9 improved, 5/9 stable and 3/9 worse. For the entire 12-month period, 1/9 were improved, 6/9 were stable and 2/9 worse.

HRCT scans done on 9/10 subjects 12 months after the start of treatment were compared with those done on entry to treatment. No evidence of progression was noted in five and only slight progression was noted in the other four (Table 4).

Seven subjects were given the retrospective questionnaire about their IPF-associated cough. Five reported an early marked improvement; one reported a worsening; and one did not have a cough on entry (Table 5).

TABLE 2
OUTCOMES FOR FVC (% PREDICTED) FOR INDIVIDUAL
SUBJECTS OVER TWO CONSECUTIVE SIX-MONTH PERIODS

| | | Predicted* | Actual | Outcome** | Predicted* | Actual | Outcome** | Predicted* | Outcome |
|---|---|---|---|---|---|---|---|---|---|
| | Baseline | Value at | Value at | for First | Value at | Value at | for Second | Value for | for |
| Subject | Value | 6 months | 6 months | Period | 12 months | 12 months | Period | One Year | One Year |
| 1 | 49.8 | 44.8 | 52.5 | S | 47.2 | 52.2 | S | 40.3 | S |
| 3 | 36.7 | 33.0 | 38.5 | S | 34.6 | 41.8 | S | 29.4 | I |
| 4 | 73.4 | 66.1 | 70.3 | S | 63.3 | 72.7 | S | 59.4 | S |
| 5 | 59.0 | 53.1 | 59.8 | S | 53.8 | 51.0 | W | 47.8 | W |
| 8 | 72.6 | 65.3 | 69.1 | S | 62.2 | 67.4 | S | 58.8 | S |
| 11 | 47.3 | 42.6 | 37.7 | W | 33.9 | 37.4 | S | 38.3 | W |
| 12 | 66.0 | 59.4 | 78.8 | I | 70.9 | 72.3 | S | 53.5 | S |
| 15 | 54.0 | 48.6 | 63.4 | I | 57.1 | 56.7 | W | 43.7 | S |
| 16 | 43.1 | 38.8 | 40.0 | S | 36.0 | 40.0 | S | 34.9 | S |
| 18 | 68.3 | 61.5 | 62.2 | S | 56.0 | 59.7 | S | 55.3 | W |

*Lower limit for a stable outcome based on the range of +/−10% change over 6 months. Value for each subject at 6 months used as the baseline for the second period.
**S = stable, W = worse, I = improved. Outcome for one year assessed by comparing value at 12 months with value for maximal decrease in stable range computed by 0.9x − (0.1)(0.9x) where baseline = x

TABLE 3
OUTCOMES FOR 02 SAT POST-EXERCISE (%) FOR INDIVIDUAL
SUBJECTS OVER TWO CONSECUTIVE SIX-MONTH PERIODS

| | | Predicted* | Actual | Outcome** | Predicted* | Actual | Outcome** | Predicted* | Outcome |
|---|---|---|---|---|---|---|---|---|---|
| | Baseline | Value at | Value at | for First | Value at | Value at | for Second | Value for | for |
| Subject | Value | 6 months | 6 months | Period | 12 months | 12 months | Period | One Year | One Year |
| 1 | 75 | 72 | 75 | S | 72 | 75 | S | 69 | S |
| 3 | 85 | 82 | 78 | W | 75 | 82 | I | 78 | S |
| 4 | 99 | 95 | 96 | S | 92 | 97 | S | 91 | S |
| 5 | 61*** | 60 | 63 | S | 60 | 60 | S | 58 | S |
| 8 | 91*** | 89 | 93 | S | 89 | 92 | S | 85 | S |
| 11 | Subject unable to exercise due to physical handicap | | | | | | | | |
| 12 | 83 | 80 | 83 | S | 80 | 82 | S | 76 | S |
| 15 | 80 | 77 | 74 | W | 71 | 65 | W | 74 | W |
| 16 | 67 | 64 | 82 | I | 79 | 77 | W | 62 | I |
| 18 | 83 | 80 | 82 | S | 79 | 73 | W | 76 | W |

TABLE 4
OBSERVATIONS ON HRCTs

| Subject: | Treatment Period: | Changes Observed: |
|---|---|---|
| 01 | 12 Months | No progression |
| 03 | 12 Months | No progression |
| 04 | 48 Months | No progression |
| 05 | 12 Months | Slight progression |
| 08 | 12 Months | Slight progression |
| 11 | 12 Months | One year scan not done |
| 12 | 24 Months | No progression |
| 15 | 12 Months | Slight progression |
| 16 | 12 Months | Slight progression |
| 18 | 12 Months | No progression |

TABLE 5
EFFECT OF TREATMENT ON IPF-RELATED COUGH
BASED ON A RETROSPECTIVE QUESTIONNAIRE*

| | Overall | Duration of | Intensity of | Frequency | Change in |
|---|---|---|---|---|---|
| | Change in | Cough | Cough | of Cough | Quality of |
| Subject | Cough | Episodes | Episodes | Episodes | Life |
| 12 | Better | −4 | −3 | −4 | +4 |
| 15 | Better | −4 | Same | Same | +3 |
| 16 | Better | Same | −4 | −4 | +3 |
| 18 | Worse | −4 | Same | +4 | −1 |
| 19 | Better | −3 | −2 | −2 | +3 |
| 20 | Better | −2 | −1 | −4 | +4 |
| | | — | | | |

*Changes in cough status after treatment for 1 month rated on scale of 0 to 4 where 0 = none & 4 = large; + = increase; − = decrease; Same = no change
** Subject 4 had no history of IPF-related cough. Subjects 19 & 20 have not yet completed one year of treatment and therefore, were not included in the analysis of pulmonary function.

### CONCLUSIONS

Treatment with low-dose, orally-administered interferon-alpha (150 IU tid) was well tolerated by IPF patients with no side effects being reported, consistent with what has been observed in previous clinical trials in other diseases. Preliminary retrospective data strongly suggest that this regimen in most subjects leads to a rapid and significant reduction in the cough associated with IPF, resulting in an improvement in the quality of life.

All subjects had severely compromised lung function on study entry. Most of the ten subjects treated for a 12-month period had stable or minimally progressive disease using criteria suggested in the International Consensus Statement. However, assessment by spirometry, pulse oximetry and HRCT was discordant in some cases. It is noteworthy that two subjects, still under treatment, have shown no progression over 24 and 48 months, respectively, assessing all variables.

Each of the subjects served as their own control, and there was no placebo control group for comparison. However, given the well-documented rapid rate of progression and the life expectancy after diagnosis of between 3.2 and 5 years, stability over this long a period of time in this high a percentage of subjects strongly suggests potential efficacy of this treatment regimen.

Accordingly, the data indicate that treatment with low-dose, orally administered interferon-alpha may be an inexpensive, non-invasive and safe approach to preventing or decreasing the rate of further deterioration of lung function in patients with IPF.

### REFERENCES

1. Panos R J, R Mortenson, S A Niccoli and T E King, Jr. Clinical deterioration in patients with idiopathic pulmonary fibrosis: causes and assessment. Am. J. Med. 88:396-404, 1990.
2. International Consensus Statement: Idiopathic Pulmonary Fibrosis: Diagnosis and Treatment. Am. J. Respir. Crit. Care Med. 161:646-664, 2000.
3. Bartram U and C P Speer. The role of transforming growth factor β in lung development and disease. Chest. 125:754-765, 2004.
4. Sime P J, Z Xing, F L Graham, K G CSAKY and J Gauldie. Adenovector-mediated gene transfer of active transforming growth factor-β1 induces prolonged severe fibrosis in rat lung. J. Clin. Invest. 100:768-776, 1997.
5. Burgess H A, L E Daugherty, T H Thatcher, H F Lakatos, D M Ray, M Redonnet, R P Phipps and P J Sime. PPARγ agonists inhibit TGF-β induced pulmonary myofibroblast differentiation and collagen production: implications for therapy of lung disease. Am. J. Lung Cell Mol. Physiol. 288:L1146-L1153, 2005.
6. Ziesche R, E Hofbauer, K Wittmann, V Petkov and L Block. A preliminary study of long-term treatment with interferon Gamma-1b and low dose prednisolone in patients with idiopathic pulmonary fibrosis. New Engl. J. Med. 341:1264-1269, 1999.
7. Broekelmann, T J, A H Limper, T V Colby and J A McDonald. Transforming growth factor beta 1 is present at sites of extracellular matrix expression in human pulmonary fibrosis. Proc. Natl. Acad. Sci. USA 88:6642-6646.
8. Prior C and P L Haslam. In vivo levels and in vitro production of interferon-gamma in fibrosing interstitial lung diseases. Clin. Exp. Immunol. 88:280-287, 1992.
9. Leask A and D J Abraham. TGF-β signaling and the fibrotic response. FASEB J. 18:816-827, 2004.
10. Elias J A, S A Jimenez and B Freundlich. Recombinant gamma, alpha and beta interferon regulation of human lung fibroblast proliferation. Amer. Rev. Respir. Dis. 135:62-65, 1987.
11. Gurujeyalakshmi G and S N Giri. Molecular mechanisms of antifibrotic effect of interferon gamma in bleomycin-mouse model of lung fibrosis; downregulation of TGF-β and procollagen I and III gene expression. 21:791-808, 1995.
12. Smith J K, A Afzal, A. Siddiqui, L A Modica, R Dykes, C. Simmons, J Schmidt, G A Krishnaswamy and S L Berk. Interferon-α upregulates gene expression of aquaporin-5 in human parotid glands. J. Interferon Cytokine Res. 19:925-935, 1999.
13. Raghu G, K Brown, W. Bradford, K Starko, D. Schwartz and T King. A placebo-controlled trial of interferon gamma-1b in patients with idiopathic pulmonary fibrosis. New Engl. J. Med. 350:125-133, 2004.
14. Shiozawa S, Y Tanaka and K. Shiozawa. Single-blinded controlled trial of low-dose oral interferon-alpha for the treatment of Xerostomia in patients with Sjogren's Syndrome. J. Interferon Cytokine Res. 18:255-262, 1998.
15. Lau A S, G E Hannigan, M H Freedman and B R G Williams. Regulation of interferon receptor expression in human blood lymphocytes in vitro and during interferon therapy. J. Clin. Invest. 77:1632-1638, 1986.
16. Cummins J, G Krakowka and C Thompson. Systemic effects of interferons after oral administration in animals and humans. Amer. J. Vet. Med. 66:164-176, 2005.

### EXAMPLE 2

Orally Administered Interferon-alpha Lozenges for Treatment of IPF—Effect of Interferon-alpha Treatment on Cough in a Subset of Study Subjects

A pilot study in the treatment of IPF with orally administered Interferon-alpha lozenges is ongoing at Texas Tech University Health Sciences Center in Lubbock, Tex. Patients in this open-label trial are receiving a 150 IU Interferon-alpha lozenge 3 times per day. A total of 18 patients have been enrolled to treatment to date of whom 7 subjects remain on study. As described in Example 1, data collected to date indicate the capacity of interferon-alpha lozenge therapy to stabilize lung function in IPF patients.

While conducting the study, one of the investigators began receiving anecdotal reports from multiple subjects and subjects' family members that cough, a frequent complaint of IPF patients, had been significantly reduced or even eliminated during treatment. Since cough was not a pre-defined study endpoint, the investigator devised a new two-part questionnaire to administer to the ongoing subjects in order to retrospectively evaluate the effect of interferon-alpha lozenge therapy on cough in IPF patients.

The first part of the questionnaire asked the subjects to recall various aspects of their cough prior to starting treatment, such as cough frequency, duration, and severity as well as the extent to which coughing disrupted sleep and impacted quality of life (QOL). The second part of the questionnaire asked the subjects to report on these same aspects of their cough after 3 months on treatment and to report on changes from baseline. Of the 7 ongoing subjects, one reported no cough. The following tables summarize the questionnaire responses received from the remaining 6 subjects who reported a history of coughing prior to beginning interferon-alpha treatment in this study. Each table reports on 6 subjects except where noted.

TABLE 6

Presence of
coughing spells (no. of subjects)

| | Before interferon | After interferon |
|---|---|---|
| Yes | 6 | 5 |
| No | 0 | 1 |

TABLE 7

Duration of coughing
spells (minutes)

| | Before interferon | After interferon |
|---|---|---|
| Highest duration | Constant | 5–10 |
| Lowest duration | 1–2 | 0 |
| Median duration | 13.25 | 2.75 |

TABLE 8

| | Change in coughing spell |
|---|---|
| | duration (no. of subjects) |
| | After interferon |
| Decreased | 4 |
| Increased | 1 |
| Unchanged | 1 |

TABLE 9

Coughing spell extent
(0–4 pt. scale)*

| | Before interferon | After interferon |
|---|---|---|
| Highest score | 4 | 4 |
| Lowest score | 2 | 0 |
| Median score | 3 | 1 |

*Scale ranges from 0 = none or best possible to 4 = severe or worst possible. The same scale is used in the following tables except where noted.

TABLE 10

Number of coughing spells
per day

| | Before interferon | After interferon |
|---|---|---|
| Highest number | 10–15 | 3–4 |
| Lowest number | 1–2 | 0 |
| Median number | 4 | 1.25 |

TABLE 11

Coughing spell intensity
(0–4 pt. scale)

| | Before interferon | After interferon |
|---|---|---|
| Highest score | 4 | 3 |
| Lowest score | 2 | 0 |
| Median score | 3 | 1 |

TABLE 12

Presence of coughing spells
at night (no. of subjects)

| | Before interferon | After interferon |
|---|---|---|
| Yes | 5 | 1 |
| No | 1 | 5 |

TABLE 13

Sleep disturbed by coughing
spells (no. of subjects)
(n = 5)*

| | Before interferon | After interferon |
|---|---|---|
| Yes | 4 | 1 |
| No | 1 | 4 |

*One patient reported no coughing at night (see table 12).

TABLE 14

Amount of sleep disturbance -
subject (0–4 pt. scale)
(n = 5)*

| | Before interferon | After interferon |
|---|---|---|
| Highest score | 4 | 4 |
| Lowest score | 1 | 0 |
| Median score | 2 | 0 |

*One patient reported no coughing at night (see table 12).

TABLE 15

Amount of sleep disturbance -
partner (0–4 pt. scale)
(n = 4)*

| | Before interferon | After interferon |
|---|---|---|
| Highest score | 4 | 4 |
| Lowest score | 2 | 0 |
| Median score | 4 | 0 |

*One patient reported no coughing at night (see table 12) and another had no partner.

TABLE 16

| | Nature of change in |
|---|---|
| | coughing at 3 months |
| | (no. of subjects) |
| | After interferon |
| Better overall | 5 |
| Worse overall | 1 |

TABLE 17

Time to noticeable
change in coughing

| | Better (n = 5)* | | Worse (n = 1) |
|---|---|---|---|
| Highest duration | 4 | weeks | 2 weeks |
| Lowest duration | 5–7 | days | 2 weeks |
| Median duration | 2–3 | weeks | — |

*Tables 17–21 contrast the 5 patients who reported overall improvement in cough with the one patient who reported overall worsening of cough.

TABLE 18

Overall change in
coughing after treatment
(0–4 pt. scale)*

| | Better (n = 5) | Worse (n = 1) |
|---|---|---|
| Highest score | 4 | 2 |
| Lowest score | 3 | 2 |
| Median score | 3 | — |

*For the patients in the “better” column, scores indicate the amount of improvement.

TABLE 19

Change in daytime
coughing after treatment
(0–4 pt. scale)*

| | Better (n = 5) | Worse (n = 1) |
|---|---|---|
| Highest score | 4 | 2 |
| Lowest score | 3 | 2 |
| Median score | 3 | — |

*For the patients in the “better” column, scores indicate the amount of improvement.

TABLE 20

Change in nighttime
coughing after treatment
(0–4 pt. scale)*

| | Better (n = 4)** | Worse (n = 1) |
|---|---|---|
| Highest score | 4 | 3 |
| Lowest score | 0 | 3 |
| Median score | 4 | — |

*For the patients in the “better” column, scores indicate the amount of improvement.
**One patient reported no coughing at night (see table 12).

TABLE 21

Impact of change
in cough on overall
QOL (0–4 pt. scale)*

| | Better (n = 5) | Worse (n = 1) |
|---|---|---|
| Highest score | 4 | 1 |
| Lowest score | 3 | 1 |
| Median score | 3 | — |

*For the patients in the “better” column, scores indicate the amount of improvement.

All 6 patients reported a noticeable change in their cough within 1 to 4 weeks after starting interferon-alpha lozenge therapy in this study. Five subjects reported an improvement in their cough, including one subject who reported a cessation of coughing. A single subject thought his cough was worse.

Median duration of coughing was reduced from 13.25 minutes at baseline to 2.75 minutes after treatment, and the median number of coughing spells per day was reduced from 4 to 1.25. Extent and intensity of coughing were both reduced from a median score of 3 (“a lot”) at baseline to a median score of 1 (“some”) following initiation of interferon-alpha treatment.

Reduction in nighttime coughing was a particular benefit evident in this study. Of the 5 patients who complained of nighttime coughing at baseline, 4 reported cessation of this symptom (as well as elimination of sleep disturbance caused by coughing) after receiving interferon-alpha treatment. Of these 4 patients, the 3 who indicated that their nighttime coughing at baseline disturbed their partner's sleep reported an elimination of this problem after starting interferon-alpha therapy.

Improvement in coughing symptoms had an apparent beneficial effect on overall QOL in this study. Of the 5 IPF patients who thought their coughing was improved in response to interferon-alpha treatment, 3 subjects indicated that their QOL was improved “a lot,” and the other 2 subjects indicated that their QOL was improved to the maximal degree. The single patient who thought his cough was worse after starting interferon-alpha therapy indicated that his QOL was impacted negatively to “some” degree.

Changes from baseline were calculated from the individual patient questionnaire responses and summarized in Table 22. With one exception, negative scores and percentages indicate improvement (i.e., the symptom was reduced), while positive scores/percentages indicate worsening of a symptom. The reverse is true for QOL, for which an increase in score indicates an improvement.

TABLE 22
Summary of symptom changes by patient

| | Patient | | | | | |
|---|---|---|---|---|---|---|
| | A | C* | D | E | F | G |
| Overall change | Better | Better | Worse | Better | Better | Better |
| in cough | | | | | | |
| Duration | +50% | −67% | −89% | −99% | −100% | Same |
| Extent | −2 | −2 | ND | −1 | −3 | Same |
| Frequency | −85% | −67% | +133% | Same | −100% | −88% |
| Intensity | −1 | −2 | Same | Same | −3 | −1 |
| Sleep | −1 | NA | Same | −4 | −2 | −1 |
| disturbance, | | | | | | |
| self | | | | | | |
| Sleep | −2 | NA | Same | −4 | −4 | NA |
| disturbance, | | | | | | |
| partner | | | | | | |
| Quality of life | +4 | +3 | −1 | +3 | +4 | +3 |

*Subject B reported no history of coughing, so his questionnaire responses were excluded from this report.

In conclusion, results of a retrospective analysis in 6 IPF patients with a history of cough in an ongoing, open-label study indicate that orally administered interferon-alpha lozenge therapy has an apparently beneficial effect on cough. Five of 6 patients reported an overall improvement in their cough after 3 months of treatment, with corresponding increases in QOL. Duration, frequency and severity of cough were reduced in 4 of 6 patients each. Nighttime coughing was eliminated in 4 of 5 patients with a history of this complaint, which had the beneficial effect of eliminating sleep disturbance for the subjects and their partners.

### EXAMPLE 3

Reduction of Chronic Cough in Patients with Sjögren's Syndrome

Sjögren's syndrome is a chronic disorder that causes damage to the salivary glands, resulting in dry mouth, and the tear glands, resulting in dry eyes. It can also affect other parts of the body including joints, muscles and nerves, organs such as the lungs, kidneys, liver, pancreas, stomach and brain, or glands such as the thyroid gland. Symptoms associated with Sjogren's syndrome include dry eyes/mouth, swelling, difficulty chewing or swallowing, dry cough, cavities, oral yeast infections, dry nose, throat and lungs and fatigue.

Chronic dry cough is a symptom that affects some individuals afflicted with Sjogren's syndrome. Surprisingly, applicants have found that the oral administration of interferon-alpha has provided relief to two individuals who were diagnosed with Sjogren's syndrome and had noted their discomfort from coughing. Each of these individuals, while receiving interferon to treat the dry eyes/mouth symptoms, noted an improvement in regards to their coughing. One patient receiving an oral dose of interferon-alpha, in lozenge form 2× a day for the past 5 years, reported that “the medicine did and does help the cough. . . . ” The second patient also expressed improvement in controlling her cough.

All references, including publications, patent applications, and patents, cited herein are hereby incorporated by reference to the same extent as if each reference were individually and specifically indicated to be incorporated by reference and were set forth in its entirety herein.

The use of the terms “a,” “an,” “the,” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate better the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. It should be understood that the illustrated embodiments are exemplary only, and should not be taken as limiting the scope of the invention.

1. A method of treating chronic coughing in a patient, said method comprising administering to the patient interferon in an amount effective to diminish a cough characteristic selected from the group consisting of cough duration, cough frequency and cough intensity.

2. The method of claim 1 wherein the interferon is a type I interferon.

3. The method of claim 1 wherein the interferon is administered orally.

4. The method of claim 1 wherein the interferon is administered bucally.

5. The method of claim 1 wherein the interferon is administered topically to the lung cells by inhalation.

6. The method of claim 5 wherein the interferon is interferon-alpha that is administered using a metered dose inhaler.

7. The method of claim 6 wherein a metered dose from the metered dose inhaler is from about 75 IU to about 1,000 IU of interferon-alpha.

8. The method of claim 3 wherein the interferon is interferon-alpha and the effective amount administered is from about 0.1 IU/lb to about 100 IU/lb of patient body weight.

9. A method of treating idiopathic pulmonary fibrosis in a patient, said method comprising orally administering to the patient a composition comprising interferon.

10. The method of claim 9 wherein the interferon is interferon-alpha that is administered in a dosage form of about 0.1 IU/lb to about 100 IU/lb of patient body weight.

11. The method of claim 9 wherein the interferon is interferon-alpha that is administered using a metered dose inhaler.

12. The method of claim 11 wherein a metered dose from the metered dose inhaler is from about 75 IU to about 1,000 IU of interferon-alpha.

13. The method of claim 9 wherein the interferon is administered in an amount effective to diminish a cough characteristic selected from the group consisting of cough duration, cough frequency and cough intensity.
